(19) United States
(12) Patent Application Publication
(10) Pub. No.: US 2024/0344904 A1
(43) Pub. Date: Oct. 17, 2024

(54) TEMPERATURE MEASUREMENT DEVICE

(71) Applicant: Nippon Telegraph and Telephone Corporation, Tokyo (JP)

(72) Inventors: Daichi Matsunaga, Tokyo (JP); Yujiro Tanaka, Tokyo (JP); Takuro Tajima, Tokyo (JP); Michiko Seyama, Tokyo (JP)

(21) Appl. No.: 18/682,630

(22) PCT Filed: Aug. 12, 2021
(86) PCT No.: PCT/JP2021/029723

§ 371 Date: Feb. 9, 2024

(51) Int. Cl. G01K 17/16; G01K 13/20

(52) U.S. Cl. CPC G01K 17/16; G01K 13/20

(57) ABSTRACT

This temperature measurement device includes a temperature sensing unit serving as a detector for measuring a magnitude of a heat flow transmitted from a living body, a housing having a hollow structure, a heat flow compensation mechanism with a hollow structure that is disposed in a space inside the housing so as to cover the temperature sensing unit and transports a heat flux from the living body outside the temperature sensing unit to an upper part of the temperature sensing unit, and a circuit board mounted on the heat flow compensation mechanism. The circuit board includes a processing electronic circuit for calculating the internal temperature of the living body on the basis of the magnitude of the heat flow measured by the temperature sensing unit.

### CROSS-REFERENCE TO RELATED APPLICATIONS

This application is a national phase entry of PCT Application No. PCT/JP2021/029723, filed on Aug. 12, 2021, which application is hereby incorporated herein by reference.

### TECHNICAL FIELD

The present invention relates to a temperature measurement device for measuring the internal temperature of a living body and the like.

### BACKGROUND

A device for non-invasively measuring the internal temperature (core body temperature) of a living body has been proposed (see PTL 1). The technique disclosed in PTL 1 estimates a core body temperature T_{CBT} of a living body 100 by using a thermal equivalent circuit model of the living body 100 and a temperature measurement device 101 as shown in FIG. 8. Reference numeral 102 shown in FIG. 8 represents outside air. The core body temperature T_{CBT} is calculated by the following formula.

$${T}_{CBT} = {T}_{1} + A \times H \qquad ( 1 )$$

T₁ is the temperature of the skin surface of the living body 100, A is the proportionality coefficient, and H is the magnitude of the heat flow being measured. The magnitude H of the heat flow is expressed by the difference between the temperatures T₁ and T₂ as shown in the following formula.

$$H = {T}_{1} - {T}_{2} \qquad ( 2 )$$

T₂ is the temperature of the upper surface of the temperature measurement device 101 on the opposite side to the surface in contact with the living body 100. The proportionality coefficient A can be obtained as follows by substituting the temperature of the tympanic membrane measured by a tympanic membrane thermometer at the start of measurement or during measurement or the rectum temperature measured by a rectum thermometer, into the equation (1) as T_{CBT}.

$$A = {R}_{Body} / {R}_{S e n s o r} = ( {T}_{CBT} - {T}_{1} ) / H \qquad ( 3 )$$

R_{Body} is a thermal resistance of the living body 100, and R_{Sensor} is a thermal resistance of the temperature measurement device 101. According to the technique disclosed in PTL 1, when the convection of the outside air becomes strong, the heat to be introduced into a temperature sensing unit of the temperature measurement device 101 flows out to the outside air as shown by, for example, reference numeral 103 in FIG. 9, and the magnitude of the heat flow to be originally measured decreases from H to H′ (H′<H). Therefore, there is a problem that an error occurs in estimation of the core body temperature T_{CBT}.

In order to reduce the estimation error of the core body temperature T_{CBT}, a method of providing a cover made of a material having a high thermal conductivity inside the temperature measurement device 101 can be considered. Hereinafter, such a cover is referred to as a heat flow compensation mechanism. By providing the heat flow compensating mechanism 104 inside the temperature measurement device 101, it is considered that the heat flow changes from 103 shown in FIGS. 9 to 105 shown in FIG. 10. Thus, the difference between the magnitude H′ of the heat flow to be measured and the true value H can be reduced, and the estimation error of the core body temperature T_{CBT} can be reduced.

However, in the actual temperature measurement device 101, a circuit board for mounting a processing electronic circuit and a battery or the like is required in addition to a temperature sensing unit for measuring the temperatures T₁ and T₂. The processing electronic circuit calculates the value of the core body temperature T_{CBT} from the temperatures T₁, T₂ and transmits the value to the outside. In the case where the circuit board is incorporated into the temperature measurement device 101, it is necessary to separate the housing for accommodating the temperature sensing unit and the heat flow compensation mechanism from the housing for accommodating the circuit board, in order to prevent the heat generation of the processing electronic circuit from affecting the heat flow to the temperature sensing unit. In a case where the housings are divided in this manner, when the temperature measurement device 101 is used as a wearable device to be attached to the living body, the area of the temperature measurement device that is mounted onto the living body is increased. Furthermore, since the housings are electrically connected to each other via wiring, handling becomes difficult, resulting in reduced convenience.

### CITATION LIST

### Patent Literature

PTL 1-Japanese Patent Application Publication No. 2020-003291

### SUMMARY

### Technical Problem

Embodiments were contrived in order to solve the foregoing problems, and an object thereof is to provide a compact, easy-to-handle temperature measurement device capable of accurately measuring the internal temperature of a living body even when the convection state of the outside air changes.

### Solution to Problem

A temperature measurement device of embodiments of the present invention includes: a detection unit configured to measure a magnitude of a heat flow transmitted from a living body; a housing with a hollow structure that covers the detection unit and forms a space between the housing and the detection unit; a heat flow compensation mechanism with a hollow structure that is disposed in a space inside the housing so as to cover the detection unit and is configured to transport a heat flux from the living body outside the detection unit to an upper part of the detection unit; and a circuit board mounted on the heat flow compensation mechanism, wherein the circuit board includes an electronic circuit configured to calculate an internal temperature of the living body on the basis of a magnitude of the heat flow measured by the detection unit.

In one configuration example of the temperature measurement device according to embodiments of the present invention, the heat flow compensation mechanism has a frustum shape in which an area of a top surface separated from the living body is smaller than an area of a bottom surface on the living body side.

In one configuration example of the temperature measurement device according to embodiments of the present invention, the detection unit is arranged so as to be in contact with an inner wall of the top surface of the heat flow compensation mechanism.

In one configuration example of the temperature measurement device, the detection unit is arranged at a position near a center line of the frustum of the heat flow compensation mechanism.

In one configuration example of the temperature measurement device according to embodiments of the present invention, the electronic circuit is arranged on the circuit board at a distance from a position through which the center line of the frustum of the heat flow compensation mechanism passes.

One configuration example of the temperature measurement device according to embodiments of the present invention further comprises a battery configured to supply a power supply voltage to the electronic circuit, wherein the battery is arranged in the vicinity of the position through which the center line of the frustum of the heat flow compensation mechanism passes.

In one configuration example of the temperature measurement device according to embodiments of the present invention, the detection unit includes a first temperature sensor configured to measure a first temperature of a skin surface of the living body, a second temperature sensor configured to measure a second temperature at a position away from the living body, and a fixing member for holding the first and second temperature sensors, wherein a difference between the first temperature and the second temperature is measured as a magnitude of a heat flow transmitted from the living body.

In one configuration example of the temperature measurement device according to embodiments of the present invention, a ratio H2/ΦD of a diameter ΦD of the heat flow compensation mechanism to a height H2 of a space existing on the circuit board in the housing is less than 0.2.

### Advantageous Effects of Embodiments of the Invention

According to embodiments of the present invention, by providing the heat flow compensation mechanism, even when the convection state of the outside air changes, the difference between the magnitude of the heat flow to be measured and the true value thereof can be reduced, and the estimation error of the internal temperature of the living body can be reduced. In addition, in comparison with a configuration in which the detection unit and the circuit board are incorporated in the same housing and the housing is divided by mounting the circuit board on the heat flow compensation mechanism, the area of the temperature measurement device that is mounted onto the living body can be reduced. In embodiments of the present invention, since the number of housings is one, a small-sized and easy-to-handle temperature measurement device can be realized.

### BRIEF DESCRIPTION OF THE DRAWINGS

FIG. 1 is a cross-sectional view of a temperature measurement device according to an example of the present invention.

FIG. 2 is a perspective cross-sectional view of a heat flow compensation mechanism according to an example of the present invention.

FIG. 3 is a block diagram showing an electrical configuration of the temperature estimation device according to an example of the present invention.

FIG. 4 is a diagram for explaining the effects of the temperature measurement device according to an example of the present invention.

FIG. 5 is a plan view of the temperature measurement device in which a housing for accommodating a temperature sensing unit and the heat flow compensation mechanism and a housing for accommodating a circuit board are separated from each other.

FIG. 6 is a plan view of the temperature measurement device according to an example of the present invention.

FIG. 7 is a block diagram showing a configuration example of a computer that implements the temperature measurement device according to an example of the present invention.

FIG. 8 is a diagram showing a thermal equivalent circuit model of a living body and a temperature measurement device.

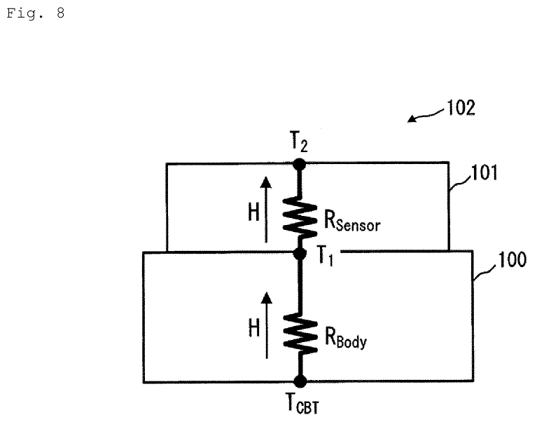

FIG. 9 is a diagram for explaining problems with a related temperature measurement device.

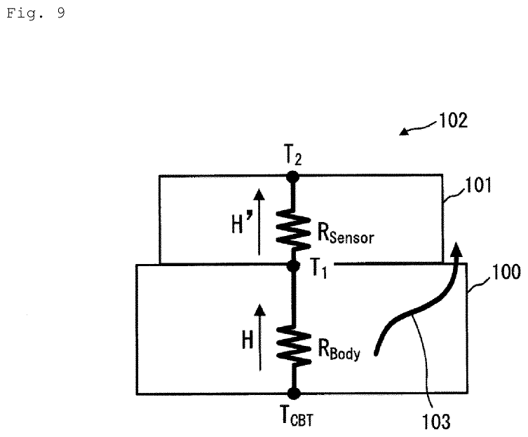

FIG. 10 is a cross-sectional view showing a configuration in which the heat flow compensation mechanism is provided inside the temperature measurement device.

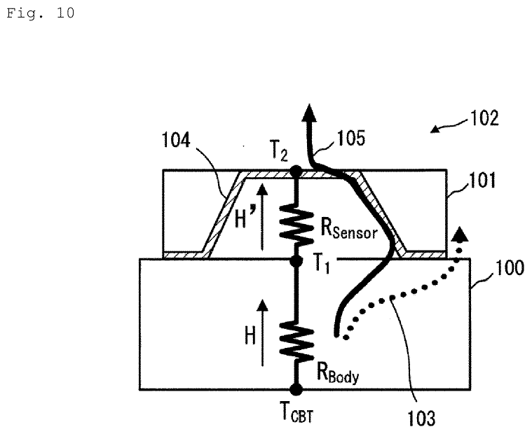

### DETAILED DESCRIPTION OF ILLUSTRATIVE EMBODIMENTS

### Principles of Embodiments of the Invention

In embodiments of the present invention, a temperature sensing unit and a circuit board are integrated in the same housing so as not to disturb the functions of a heat flow compensation mechanism, thereby reducing the area of the temperature measurement device. Since the circuit board cannot be integrated on the same surface as the temperature sensing unit in order to provide the heat flow compensation mechanism so as to surround the temperature sensing unit, a new space is provided on an upper part of the temperature sensing unit to integrate the circuit board.

### Example

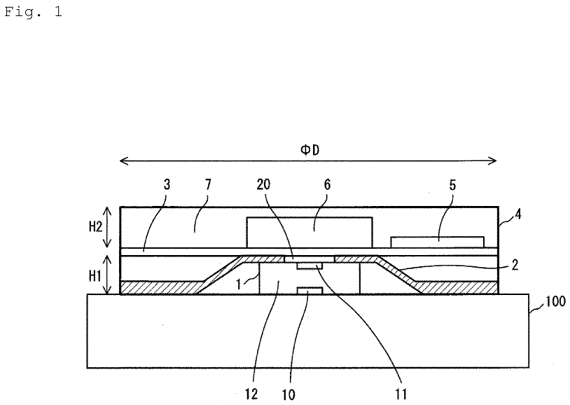

Referring to the drawings, the present invention will be described with examples. FIG. 1 is a cross-sectional view of a temperature measurement device according to an example of the present invention. The temperature measurement device includes a temperature sensing unit 1 serving as a detection unit for measuring the magnitude of a heat flow transmitted from a living body 100, a heat flow compensation mechanism 2 with a hollow structure that is arranged so as to cover the temperature sensing unit 1 and transports a heat flux from the living body 100 outside the temperature sensing unit 1 to the upper part of the temperature sensing unit 1, a circuit board 3 mounted on the heat flow compensation mechanism 2, and a housing 4 for housing the temperature sensing unit 1, the heat flow compensation mechanism 2, and the circuit board 3.

The housing 4 has a hollow structure and the inside thereof is filled with a material with high thermal resistance, that is, specifically, air. A material that has small thermal resistance and is thin is preferable as the material of the housing 4, and, for example, polyethylene terephthalate (PET) or the like can be used.

The temperature measurement device of the present example is mounted in such a manner that the temperature sensing unit 1 exposed on the surface of the housing 4 is in contact with the skin of the living body 100. It is desirable to mount the temperature measurement device on the living body 100 by using a double-sided tape or silicone rubber excellent in biocompatibility.

The temperature sensing unit 1 is composed of a temperature sensor 10 for measuring a temperature T₁ of the skin surface by contacting with the skin of the living body 100, a temperature sensor 11 for measuring a temperature T₂ of a position away from the living body 100, and a fixing member 12 in, for example, a cylindrical shape for holding the temperature sensors 10, 11. For example, thermistors, thermocouples, platinum resistors, IC (Integrated Circuit) temperature sensors, or the like can be used as the temperature sensors 10, 11.

The temperature sensor 11 is disposed immediately above the temperature sensor 10. The larger the interval between the two temperature sensors 10 and 11 is, the higher the sensitivity of the temperature measurement device. When the relative error between the two temperature sensors 10 and 11 (the difference between the values output by the temperature sensors 10 and 11 for the same temperature) is 1/100° C. or less, the interval between the temperature sensors 10 and 11 is desirably 1.5 to 4.5 mm.

When the interval between the temperature sensors 10 and 11 is changed during measurement, a proportionality coefficient A is changed, and an error is generated in estimation of a core body temperature T_{CBT} of the living body 100. Thus, the temperature sensors 10, 11 are held by using the fixing member 12. The fixing member 12 is preferably made of a hard-to-deform material such as a hard resin. The lower the thermal conductivity of the fixing member 12, the higher the sensitivity of the temperature measurement device. However, in consideration of leakage of heat to an air layer within the heat flow compensation mechanism 2, a material having a thermal conductivity of 0.1 to 1.0 [W/mK] is preferably use as the material of the fixing member 12.

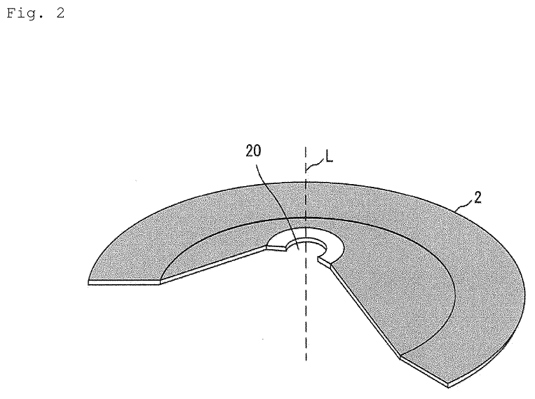

The temperature sensing unit 1 is shielded from the outside air by the heat flow compensation mechanism 2 which is a hollow structure with a conical trapezoidal shape in external form. FIG. 2 is a partially cutaway perspective cross-sectional view of the heat flow compensation mechanism 2. The heat flow compensation mechanism 2 has a frustum shape in which the area of the top surface separated from the living body 100 is smaller than the area of the bottom surface on the living body 100 side. In the heat flow compensation mechanism 2, the bottom surface on the living body 100 side is fixed to the housing 4. It is preferred that the material constituting the heat flow compensation mechanism 2 have a high thermal conductivity to efficiently transport a heat flux. For example, the heat flow compensation mechanism 2 can be configured by using a thin film made of aluminum or the like. As shown in FIGS. 1 and 2, a through-hole 20 may be formed in the top surface of the heat flow compensation mechanism 2.

The temperature sensing unit 1 is provided so that the upper surface of the fixing member 12 is brought into contact with an inner wall of the top surface of the heat flow compensation mechanism 2. As described above, when the interval between the temperature sensors 10 and 11 is 1.5 to 4.5 mm, the height H1 of the heat flow compensation mechanism 2 is, for example, 2 to 5 mm. When the through-hole 20 is provided in the top surface of the heat flow compensation mechanism 2, the temperature sensing unit 1 comes into contact with the heat flow compensation mechanism 2 at the peripheral part of the through-hole 20. It is desirable that the surface of the upper surface of the fixing member 12 be polished in order to reduce the contact thermal resistance with the heat flow compensation mechanism 2. A heat conductive sheet may be sandwiched between the upper surface of the fixing member 12 and the heat flow compensation mechanism 2.

When the heat flow compensation mechanism 2 is sufficiently larger than the temperature sensing unit 1, since the bottom surface of the heat flow compensation mechanism 2 is arranged at a position sufficiently separated from the temperature sensing unit 1, heat flux from the living body 100 is collected by the heat flow compensation mechanism 2 outside the temperature sensing unit 1 and transported to the upper part of the temperature sensing unit 1. Thus, the heat flow compensation mechanism 2 increases the temperature of the upper part of the temperature sensing unit 1 by transporting the heat flux from the living body 100 upward outside the temperature sensing unit 1, and functions to suppress the heat flux from deviating from the temperature sensing unit 1 and flowing out to the outside air.

In the heat flow compensation mechanism 2, the effects of suppressing the heat flux from deviating from the temperature sensing unit 1 and flowing out to the outside air is highest at a position near the center line (L in FIG. 2). Therefore, it is desirable to arrange the temperature sensing unit 1 near the center line L of the heat flow compensation mechanism 2. Specifically, it is desirable that the positional deviation of the temperature sensing unit 1 (temperature sensors 10, 11) from the center line L is set to, for example, 2 mm or less.

In the present example, the heat flow compensation mechanism 2 is formed in a conical trapezoidal shape corresponding to the housing 4 with a cylindrical outer shape having a hollow structure. However, the heat flow compensation mechanism 2 is not limited to a conical trapezoidal shape, as long as the shape is capable of performing the functions described above; various shapes can be adopted. For example, in the case of the housing 4 with a rectangular parallelepiped outer shape having a hollow structure, the heat flow compensation mechanism 2 can be formed into a pyramid trapezoid. By forming the heat flow compensation mechanism 2 in a conical trapezoidal shape or a pyramid trapezoid, more heat flux can be transported to the upper part of the temperature sensing unit 1, and the effect of temperature rise in the upper part of the temperature sensing unit 1 can be enhanced.

As described above, the through-hole 20 may be formed in the top surface of the heat flow compensation mechanism 2. By adjusting the size of this through-hole 20 accordingly, it is possible to adjust the depth of measurement in the case of measuring the core body temperature T_{CBT} of the living body 100. However, the provision of the through-hole 20 in the heat flow compensation mechanism 2 is not an essential component requirement of the present invention.

Next, in the present example, the circuit board 3 is mounted on the heat flow compensation mechanism 2. When the through-hole 20 is provided on the top surface of the heat flow compensation mechanism 2, the circuit board 3 comes into contact with the heat flow compensation mechanism 2 at the peripheral part of the through-hole 20. In order to reduce the contact thermal resistance between the heat flow compensation mechanism 2 and the circuit board 3, it is desirable that no circuit component be disposed in the region of the lower surface of the circuit board 3 that comes into contact with the heat flow compensation mechanism 2. When wiring exists in the region of the lower surface of the circuit board 3 that comes into contact with the heat flow compensation mechanism 2, it is desirable to protect the wiring by silk printing. The heat conductive sheet may be sandwiched between the upper surface of the heat flow compensation mechanism 2 and the lower surface of the circuit board 3.

Needless to say, it is not necessary to support the circuit board 3 by the heat flow compensation mechanism 2 alone, and the circuit board 3 may be fixed to the housing 4 as appropriate.

As described above, the heat flow compensation mechanism 2 serves to prevent the heat that should flow into the temperature sensing unit 1 from flowing out to the outside air, by transporting the heat flux from the living body 100 outside the temperature sensing unit 1 to the upper part of the temperature sensing unit 1. However, in the case where the heat flow compensation mechanism 2 does not directly contact the outside air and a space for arranging the circuit board 3 or the like is present on the heat flow compensation mechanism 2 as in the present example, heat radiation from the heat flow compensation mechanism 2 to the outside air is reduced. Therefore, the function of the heat flow compensation mechanism 2 for transporting a heat flux from the living body 100 to the upper part of the temperature sensing unit 1 and causing the heat flux to flow to the outside air is hindered, and as a result, a heat outflow to the periphery of the living body occurs, creating an error in estimation of the core body temperature T_{CBT} when the convection of the outside air changes. For this reason, the estimation error of the core body temperature T_{CBT} depends on the ratio H2/ΦD between the diameter ΦD of the heat flow compensation mechanism 2 and the height H2 of a space 7 existing on the circuit board 3 in the housing 4.

If the estimation error of the deep body temperature T_{CBT} is to be less than 0.1° C., H2/ΦD<0.2 should be used. If the estimation error is to be less than 0.2° C., H2/ΦD<0.5 should be used. If the estimation error is to be less than 0.3° C., H2/ΦD<0.8 should be used. If the estimation error is to be less than 0.4° C., H2/ΦD<1.3 should be used. If the estimation error is to be less than 0.5° C., H2/ΦD<2.0 should be used. When the diameter ΦD of the heat flow compensation mechanism 2 is 30 mm, and, for example, the estimation error is to be 0.1° C. or less, it is desirable that the height H2 of the space 7 be 6 mm or less.

In order to reduce the impact on the heat flow compensation mechanism 2, it is desirable that a processing electronic circuit 5 with high heat generation be arranged on the circuit board 3, away from the position through which the center line L of the heat flow compensation mechanism 2 passes. On the other hand, it is preferable that a battery 6, which is a component having high thermal conductivity, be disposed on the circuit board 3, in the vicinity of the position through which the center line L of the heat flow compensation mechanism 2 passes.

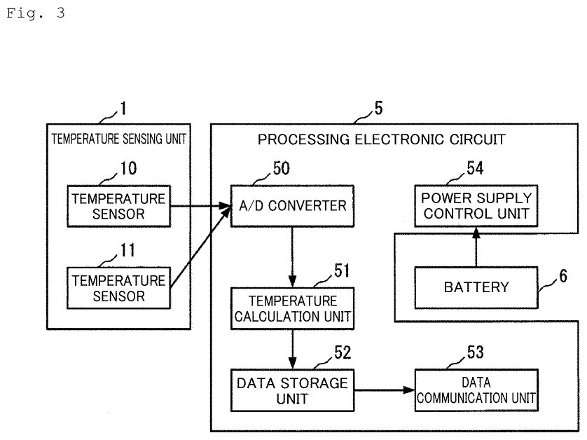

FIG. 3 is a block diagram showing an electrical configuration of the temperature measurement device of the present example. The processing electronic circuit 5 includes an AD converter 50, a temperature calculation unit 51, a data storage unit 52, a data communication unit 53, and a power supply control unit 54.

The AD converter 50 converts the temperatures T₁, T₂ measured by the temperature sensors 10, 11 into digital data.

The temperature calculation unit 51 calculates the core body temperature T_{CBT} (internal temperature) of the living body 100 by equations (1) and (2) on the basis of the temperatures T₁, T₂ and a known proportionality coefficient A.

The data storage unit 52 temporarily stores the data of the core body temperature T_{CBT} calculated by the temperature calculation unit 51.

The data communication unit 53 transmits the data of the core body temperature T_{CBT} calculated by the temperature calculation unit 51, wirelessly or wired, to an external terminal.

The power supply control unit 54 is a circuit for supplying a power supply voltage from the battery 6 to the processing electronic circuit 5.

As described above, according to the present example, by providing the heat flow compensation mechanism 2 for transporting the heat flux from the living body 100 outside the temperature sensing unit 1 to the upper part of the temperature sensing unit 1, even when the convection state of the outside air changes, the difference between the measured magnitude of heat flow (T₁−T₂) and the true value H can be reduced, and the estimation error of the core body temperature T_{CBT} can be reduced.

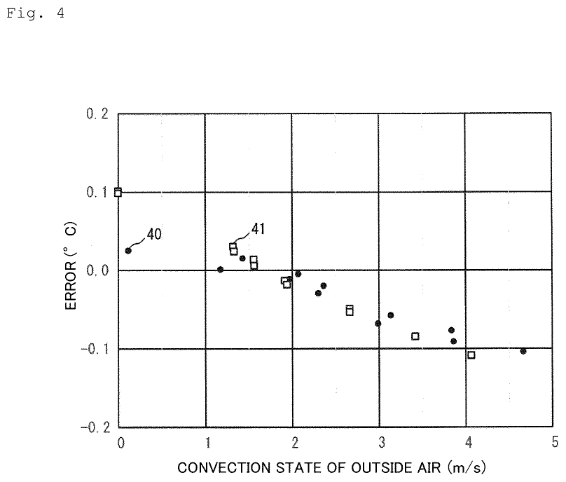

FIG. 4 is a diagram for explaining the effects of the present example. Reference numeral 40 shown in FIG. 4 indicates an estimation error of the core body temperature T_{CBT} by the temperature measurement device in which the housing for accommodating the temperature sensing unit 1 and the heat flow compensation mechanism 2 and the housing for accommodating the circuit board 3 are separated from each other in order to prevent heat generation of the processing electronic circuit 5 from affecting the heat flow to the temperature sensing unit 1. Reference numeral 41 shown in FIG. 4 indicates an estimation error of the core body temperature T_{CBT} by the temperature measurement device of the present example. In an example shown in FIG. 4, H2/ΦD<0.2 is used.

According to FIG. 4, the estimation error (±0.1° C.) equivalent to the configuration in which the housings are divided can be realized in the present example.

Further, in the present example, compared with the configuration in which the housings are divided by incorporating the temperature sensing unit 1 and the circuit board 3 in the same housing 4 and mounting the circuit board 3 on the heat flow compensation mechanism 2, the area of the temperature measurement device that is mounted onto the living body 100 can be reduced. For example, the area can be reduced to 8.6 cm² in the present example, compared to the 19.7 cm² area of the configuration that divides the housings.

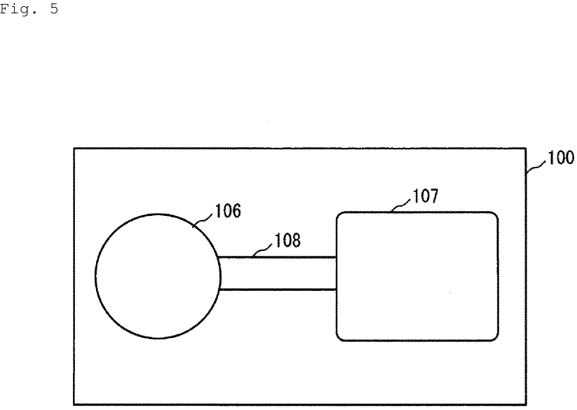

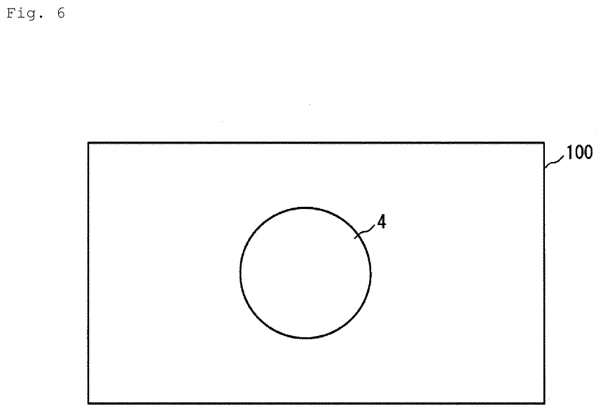

FIG. 5 is a plan view of the temperature measurement device in which the housing is divided, as viewed from above, and FIG. 6 is a plan view of the temperature measurement device of the present example. In the example shown in FIG. 5, a housing 106 for accommodating the temperature sensing unit and the heat flow compensation mechanism and a housing 107 for accommodating the circuit board are electrically connected to each other through a wiring 108. In this configuration, the area of the temperature measurement device that is mounted onto the living body 100 is increased. In addition, the separation of the housings makes it more difficult to handle and less convenient. On the other hand, according to the present example, since there is one housing, it is possible to realize a small-sized and easy-to-handle temperature measurement device.

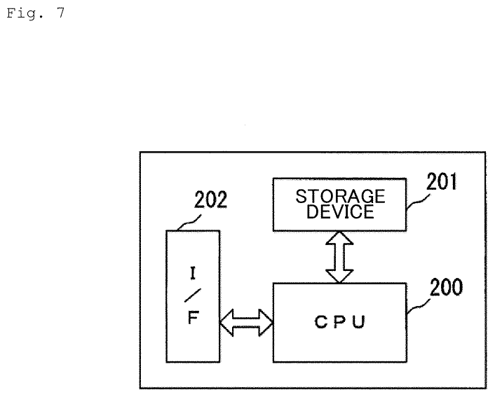

The temperature calculation unit 51, the data storage unit 52, and the data communication unit 53 described in the present example can be realized by a computer having a CPU (Central Processing Unit), a storage device, and an interface, and a program that controls these hardware resources. FIG. 7 shows a configuration example of this computer.

The computer includes a CPU 200, a storage device 201, and an interface device (I/F) 202. The temperature sensors 10, 11 and the hardware of the communication unit 53, and the like are connected to the I/F 202. In such a computer, a temperature estimation program for realizing the temperature estimation method of embodiments of the present invention is stored in the storage device 201. The CPU 200 executes the processing described in the present example according to the program stored in the storage device 201.

### INDUSTRIAL APPLICABILITY

Embodiments can be applied to techniques for non-invasively measuring the internal temperature of a living body.

### REFERENCE SIGNS LIST

1 Temperature sensing unit
2 Heat flow compensation mechanism
3 Circuit board
4 Housing
5 Processing electronic circuit
6 Battery
10, 11 Temperature sensor
12 Fixing member
20 Through-hole
50 AD converter
51 Temperature calculation unit
52 Data storage unit
53 Data communication unit
54 Power supply control unit

1-8. (canceled)

9. A temperature measurement device, comprising:
a detector configured to measure a magnitude of a heat flow transmitted from a living body;
a housing with a hollow structure that covers the detector, wherein a space is disposed between the housing and the detector;
a heat flow compensation mechanism with a hollow structure that is disposed inside the housing so as to cover the detector and is configured to transport a heat flux from the living body outside the detector to an upper part of the detector; and
a circuit board mounted on the heat flow compensation mechanism, wherein the circuit board includes an electronic circuit configured to calculate an internal temperature of the living body based on a magnitude of the heat flow measured by the detector.

10. The temperature measurement device according to claim 9, wherein the heat flow compensation mechanism has a frustum shape, and wherein a top surface of the heat flow compensation mechanism away from the living body is smaller than an area of a bottom surface the heat flow compensation mechanism towards the living body.

11. The temperature measurement device according to claim 10,
wherein the detector is in contact with an inner wall of the top surface of the heat flow compensation mechanism.

12. The temperature measurement device according to claim 10,
wherein the detector is disposed at a position of a center line of the frustum shape of the heat flow compensation mechanism.

13. The temperature measurement device according to claim 12,
wherein the electronic circuit is disposed on the circuit board, at a distance from a position through which a center line of the frustum shape of the heat flow compensation mechanism passes.

14. The temperature measurement device according to claim 13, further comprising a battery configured to supply a power supply voltage to the electronic circuit, wherein the battery is disposed on the circuit board at a position through which the center line of the frustum shape of the heat flow compensation mechanism passes.

15. The temperature measurement device according to claim 10,
wherein the electronic circuit is disposed on the circuit board, at a distance from a position through which a center line of the frustum shape of the heat flow compensation mechanism passes.

16. The temperature measurement device according to claim 15, further comprising a battery configured to supply a power supply voltage to the electronic circuit, wherein the battery is disposed on the circuit board at a position through which the center line of the frustum shape of the heat flow compensation mechanism passes.

17. The temperature measurement device according to claim 10, further comprising a battery configured to supply a power supply voltage to the electronic circuit, wherein the battery is disposed on the circuit board at a position through which a center line of the frustum shape of the heat flow compensation mechanism passes.

18. The temperature measurement device according to claim 9, wherein the detector includes:
a first temperature sensor configured to measure a first temperature of a skin surface of the living body;
a second temperature sensor configured to measure a second temperature of a location away from the living body; and
a fixing member holding the first and second temperature sensors, wherein the detector is configured to measure a difference between the first temperature and the second temperature as the magnitude of the heat flow transmitted from the living body.

19. The temperature measurement device according to claim 9,
wherein a ratio H2/ΦD between a diameter ΦD of the heat flow compensation mechanism and a height H2 of a space above the circuit board in the housing is less than 0.2.

20. A temperature measurement device, comprising:
a detector configured to measure a magnitude of a heat flow transmitted from a living body;
a hollow heat flow compensation mechanism over and covering the detector, the hollow heat flow compensation mechanism is configured to transport a heat flux from the living body outside the detector to an upper part of the detector; and
a circuit board over and mounted to the hollow heat flow compensation mechanism, wherein the circuit board includes an electronic circuit configured to calculate an internal temperature of the living body based on a magnitude of the heat flow measured by the detector.

21. The temperature measurement device according to claim 20, further comprising:
a hollow housing covering the hollow heat flow compensation mechanism and the circuit board.

22. The temperature measurement device according to claim 21,
wherein a ratio H2/OD between a diameter ΦD of the hollow heat flow compensation mechanism and a height H2 of a space above the circuit board in the hollow housing is less than 0.2.

23. The temperature measurement device according to claim 20, wherein the hollow heat flow compensation mechanism has a frustum shape, and wherein a top surface of the hollow heat flow compensation mechanism configured to be away from the living body is smaller than an area of a bottom surface the hollow heat flow compensation mechanism that configured to be attached to the living body.

24. The temperature measurement device according to claim 23,
wherein the detector is disposed at a position of a center line of the frustum shape of the hollow heat flow compensation mechanism.

25. The temperature measurement device according to claim 24,
wherein the electronic circuit is disposed on the circuit board, at a distance from a position through which a center line of the frustum shape of hollow the hollow heat flow compensation mechanism passes.

26. The temperature measurement device according to claim 24, further comprising a battery configured to supply a power supply voltage to the electronic circuit, wherein the battery is disposed on the circuit board at a position through which the center line of the frustum shape of the hollow heat flow compensation mechanism passes.

27. The temperature measurement device according to claim 20, wherein the detector includes:
a first temperature sensor configured to measure a first temperature of a skin surface of the living body;
a second temperature sensor configured to measure a second temperature of a location away from the living body; and
a fixing member holding the first and second temperature sensors, wherein the detector is configured to measure a difference between the first temperature and the second temperature as the magnitude of the heat flow transmitted from the living body.
